(19) United States
(12) Patent Application Publication
(10) Pub. No.: US 2013/0079833 A1
(43) Pub. Date: Mar. 28, 2013

(54) BONE ANCHORING ASSEMBLY

(71) Applicant: Biedermann Technologies GmbH & Co. KG, Donaueschingen (DE)

(72) Inventors: Lutz Biedermann, VS-Villingen (DE); Gerhard Pohl, St. Georgen (DE); Wilfried Matthis, Weisweil (DE)

(73) Assignee: BIEDERMANN TECHNOLOGIES GMBH & CO. KG, Donaueschingen (DE)

(21) Appl. No.: 13/627,778

(22) Filed: Sep. 26, 2012

Related U.S. Application Data
(60) Provisional application No. 61/540,239, filed on Sep. 28, 2011.

(30) Foreign Application Priority Data
Sep. 28, 2011 (EP) 11183202.8

(51) Int. Cl. A61B 17/86

(52) U.S. Cl. 606/308

(57) ABSTRACT

A bone anchoring assembly for dynamic stabilization is provided. The bone anchoring assembly includes a bone anchoring element having a head and a shank to be anchored in a bone or a vertebra, a receiving part for receiving a rod, a pressure element and a monolithic locking element. The rod (8) includes a flexible section which is made at least partly of a polymer material. The receiving part comprises a first channel with an approximately U-shaped cross-section with two free legs. Furthermore, the locking element directly cooperates with the legs to simultaneously secure the rod in the first channel (58) and exert a pressure on the pressure element that exerts pressure onto the head of the bone anchoring element to lock the angular position of the bone anchoring element relative to the receiving part. A first pin-shaped projection is provided at the locking element which comes into contact with the flexible section of the rod when tightening the locking element.

### CROSS-REFERENCE TO RELATED APPLICATIONS

The present application claims the benefit of U.S. Provisional Patent Application Ser. No. 61/540,239, filed Sep. 28, 2011, the contents of which are hereby incorporated by reference in their entirety, and claims priority to European Patent Application EP 11 183 202.8, filed Sep. 28, 2011, the contents of which are hereby incorporated by reference in their entirety.

### FIELD OF INVENTION

The invention relates to a bone anchoring assembly for dynamic stabilization comprising a bone anchoring element having a head and a shank to be anchored in a bone or a vertebra, and a receiving part for receiving a rod, the rod having a flexible section which is made at least partly of a polymer material. The receiving part comprises a first channel with an approximately U-shaped cross-section with two free legs. Furthermore, a locking element is provided directly cooperating with the legs to secure the rod in the first channel, wherein a first pin-shaped projection is provided at the locking element which comes into contact with the flexible section of the rod when tightening the locking element in such a way that in the tightened state the integral structure of the rod is not violated.

### BACKGROUND

A bone anchoring assembly with a flexible rod made of an elastomer material is known, for example, from EP 1 759 646 A1. The rod is held in the receiving part by means of a closure cap and a filling piece which presses onto the rod when the closure cap is screwed onto the receiving part. The surface of the filling piece and the bottom of the receiving part comprises conical pins which press onto the rod and create an indirect form-fit connection which contributes to the frictional connection so as to hold the rod in place. The indirect form-fit connection is achieved by a local elastic or plastic deformation of the material of the rod. The bone anchoring element is of the type of a monoaxial screw, i.e. the receiving part and the shank are not pivotably connected.

A bone anchoring assembly with a flexible rod is further known from EP 1 795 134 A1 which describes a polyaxial bone anchoring element. The receiving part and the shank are pivotably connected and a pressure element is provided to lock the angular position of the shank relative to the receiving part. The surface of the filling piece and that of the pressure element which contacts the rod have rib-like projections which press onto the flexible rod and provide a form-fit contribution to the fixation of the rod in the receiving part.

From EP 1 900 334 A1 a bone anchoring assembly of the above mentioned type is known, which comprises a single part closure element instead of a closure element with a filling piece. The single part closure element is an inner screw to be screwed between the legs of the receiving part, which has an annular projection on its lower side which presses onto the flexible rod. On the bottom of the channel of the receiving part rib-like projections are provided.

The bone anchoring assemblies mentioned above which use the flexible rod comprise an engagement structure to clamp the rod which has sharp edges and/or which has teeth or ribs which are arranged exactly on opposite sides of the rod in order to provide a safe locking.

With such engagement structures there is a risk of weakening the rod, if the rod diameter is small. Therefore, the known assemblies are mainly used with rods having a relatively large diameter, for example a diameter of approximately 9 mm or larger. However, there is a need for the use of bone anchoring assemblies of the type using a flexible rod which are small in size, in particular, when the implant is to be placed at a location which is exposed and not covered enough by muscles, ligaments or other soft tissue.

EP 2 135 574 A1 discloses a bone anchoring assembly for dynamic stabilization comprising a bone anchoring element with a shank to be anchored in a bone or a vertebra and a receiving part for receiving a rod, wherein the rod is at least partly flexible, the flexible section being made of a polymer material. The rod connects at least two bone anchoring elements. The bone anchoring element comprises a two-piece locking device for allowing a clamping of the rod in two steps.

### SUMMARY

It is an object of the invention to provide an improved bone anchoring assembly for dynamic stabilization using a flexible rod which is smaller in size compared to the known bone anchoring assemblies while providing the same degree of safe fixation of the rod as the known bone anchoring assemblies and which allows an improved handling.

The bone anchoring assembly has the advantage that it can be used, for example, with flexible elastomer rods having diameters below 9 mm. When using a small diameter rod in particular, the receiving part can also be downsized. Therefore, a low profile implant is provided, which has the advantage that the irritation of surrounding body material is small. By the one-part locking element the bone anchoring assembly has only few parts. The one-step clamping of the rod is easy, safe and effective, wherein only one tool is needed for screwing in the single locking element. Therefore, an easy and comfortable handling of the bone anchoring assembly is possible.

When the surgeon would like to connect three or more bone anchoring devices in a row via a rod, by a tube-shaped extension the rod can be easily pressed down via the inner screw even if the bone anchoring devices are mounted on different levels in the body. The rod is still located between the legs of the receiving part or the tube-shaped extension.

The pins that contribute to clamp the rod have a rounded tip. Hence, the integrity of the surface of the rod is not violated, since the pins do not scratch the structure.

Mechanical stops are provided preventing a penetration of the clamping pins into the surface of the rod due to limitation of the pressure force.

### BRIEF DESCRIPTION OF THE DRAWINGS

Further features and advantages will become apparent from the description of embodiments by means of the accompanying drawings.

In the drawings:

FIG. 1a shows a perspective exploded view of a bone anchoring assembly.

FIG. 1b shows a perspective view of the bone anchoring assembly according to FIG. 1a in an assembled state in a first configuration.

FIG. 1c shows a perspective view of the bone anchoring assembly of FIG. 1a in an assembled state in a third configuration.

FIG. 2a shows a perspective view of the bone anchoring assembly of FIG. 1a in the first configuration.

FIG. 2b shows a perspective view of the bone anchoring assembly of FIG. 1a in a second configuration.

FIG. 2c shows a perspective view of the bone anchoring assembly of FIG. 1a in the third configuration.

FIG. 2d shows a perspective view of the bone anchoring of FIG. 1a assembly in a fourth configuration.

FIG. 3a shows a first cross-sectional view of the bone anchoring assembly of FIG. 1a in the first configuration.

FIG. 3b shows the first cross-sectional view of the bone anchoring assembly of FIG. 1a in the second configuration.

FIG. 3c shows the first cross-sectional view of the bone anchoring assembly of FIG. 1a in the third configuration.

FIG. 3d shows the first cross-sectional view of the bone anchoring assembly of FIG. 1a in the fourth configuration.

FIG. 4a shows a second cross-sectional view of the bone anchoring assembly of FIG. 1a in the first configuration.

FIG. 4b shows the second cross-sectional view of the bone anchoring assembly of FIG. 1a in the second configuration.

FIG. 4c shows the second cross-sectional view of the bone anchoring assembly of FIG. 1a in the third configuration.

FIG. 4d shows the second cross-sectional view of the bone anchoring assembly of FIG. 1a in the fourth configuration.

### DETAILED DESCRIPTION

A bone anchoring assembly comprises one or more (not shown) bone anchoring devices 1 and a rod 8, wherein the bone anchoring devices 1 can be interconnected via the rod 8.

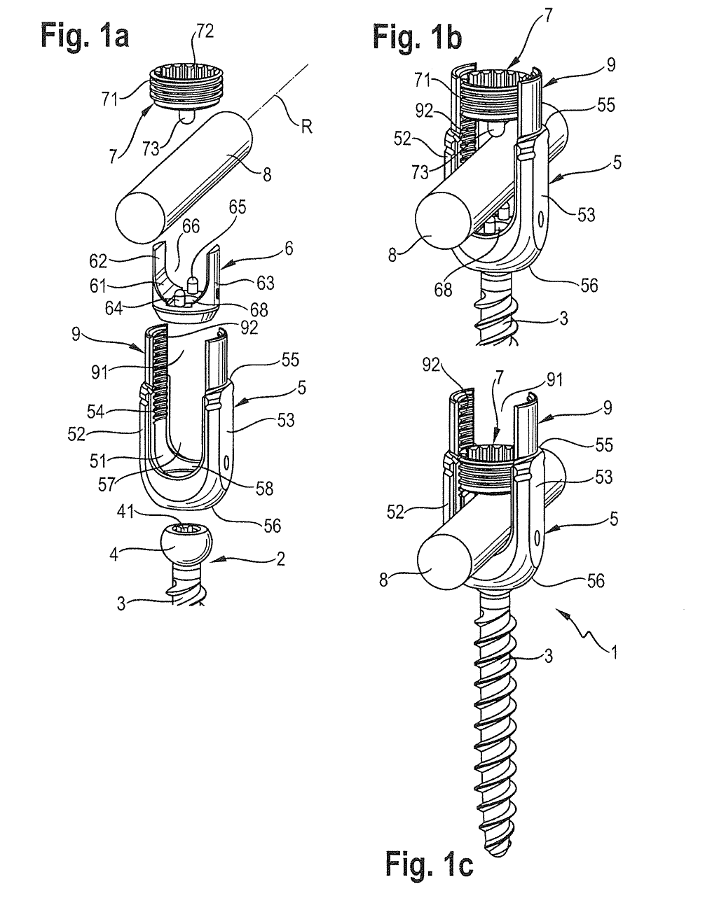

As shown in FIGS. 1a to 4d, the bone anchoring device 1 comprises a bone anchoring element 2 in the form of a polyaxial bone screw having a shank 3 with a bone thread and a tip at one end and a head 4 having an engagement structure 41 at the opposite end. The bone anchoring device 1 furthermore comprises a receiving part 5, a pressure element 6, a locking element 7 and a tube-shaped extension 9 integrally formed with the receiving part 5.

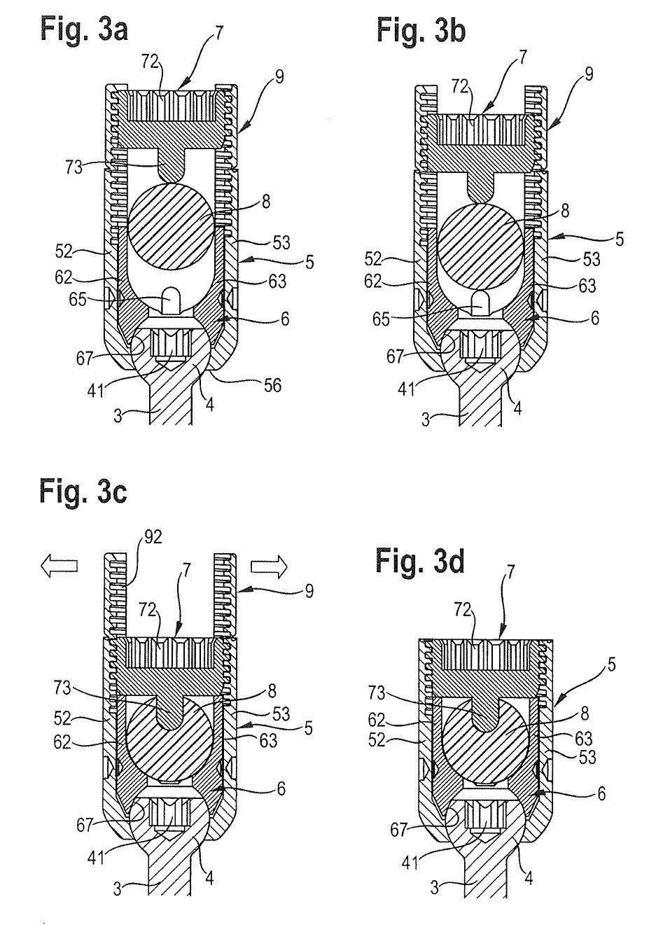

The receiving part 5 is substantially cylindrical and comprises a first end 55, a second end 56 and a coaxial bore 57 extending from the first end 55 to the second end 56 and tapering in an area near the second end 56 such that, as shown in FIG. 3a, the head 4 of the bone anchoring element 2 is pivotably held in the receiving part 5 at the second end 56. Furthermore, the receiving part 5 comprises a U-shaped recess 51 extending from the first end 55 in the direction of the second end 56. By means of the U-shaped recess 51 two free legs 52, 53 are formed which comprise an internal thread 54, wherein a channel 58 is formed.

The pressure element 6 is substantially cylindrical and dimensioned such that it can be moved within the bore 57 of the receiving part 5. The pressure element 6 has a coaxial bore 66 extending through the pressure element 6 and allowing a screwing-in tool to be guided therethrough for screwing the bone anchoring element 2 into a bone. The pressure element 6 further comprises a spherical recess 67, which is adapted to receive the spherical head 4 of the bone anchoring element 2. Further, the pressure element 6 comprises a substantially U-shaped recess 61 extending from its free end in the direction of the spherical recess 67. By means of the U-shaped recess 61 two free legs 62, 63 are formed, which form the lateral walls of a channel 68 for receiving the rod 8. On the bottom of the channel 68 two pin-shaped projections 64, 65 or pins 64, 65 are provided which are located preferably at both ends of the channel 68. The pins 64, 65 are substantially cylindrical and their free ends are rounded, preferably semi-spherical. More specifically, the pins 64, 65 are longitudinal rod-shaped pins with rounded free ends. However, they can have any shape as long as the uppermost portions are rounded as described. The uppermost portions of the pins 64, 65 are located on a line, which is parallel to the longitudinal axis R of the rod 8.

As shown in particular in FIG. 3c the pressure element 6 is sized in such a way that the legs 62, 63 of the pressure element 6 extend slightly above the surface of the rod 8 when the rod 8 is inserted into the channel 68 and the pins 64, 65 are immersed into the surface of the rod 8. The upper edge of the legs 62, 63 of the pressure element 6 form a stop for the main body of the locking element 7.

The locking element 7 is a single part locking element and can be formed as an inner screw and comprises an outer thread 71, an engagement structure 72 for engagement with a tool and a coaxial pin 73 for pressing onto the rod 8. The pin 73 is preferably cylindrical with a semi-spherical free end, more specifically, the pin 73 is a longitudinal rod-shaped pin with a rounded free end and corresponds in its dimension substantially to the dimensions of the pins 64, 65 of the pressure element 6.

The bone anchoring element 2, the receiving part 5, the pressure element 6, the locking element 7 and also the tube-shaped extension 9 can be made of a biocompatible material, such as, for example, titanium or stainless steel or another biocompatible material, for example polyether ether ketone (PEEK).

The rod 8 is made at least in part of a flexible biocompatible material, preferably of a plastic material and in particular of an elastomer material. Such a material can be based on, for example, polycarbonate-polyurethane or polycarbonate-urethane (PCU). However, other materials are also applicable, for example styrene-block-isobutylene-block-styrene (SIBS) and other elastomers. The rod 8 need not be made totally of one single material, but can comprise several materials and inner structures and/or sections with different flexibility and/or rigidity. The flexible section is the section which is to be clamped in the receiving part 5. The diameter of the rod 8 can be any of the usual smaller diameters of rods for stabilization of the spine, in particular diameters from 4.5 mm to 9 mm.

The coaxial tube-shaped extension 9 has a slot 91 for inserting the rod 8 and an internal thread 92 which cooperates with the inner thread 54 of the receiving part 5. The extension 9 is used for minimally invasive surgery and can be broken away after tightening the locking element 7, preferably manually. For breaking away the tube-shaped extension 9 in an easy way, a predetermined breaking point, i.e., a material weakness formed by a groove between the receiving part 5 and the tube-shaped extension 9 for example can be provided.

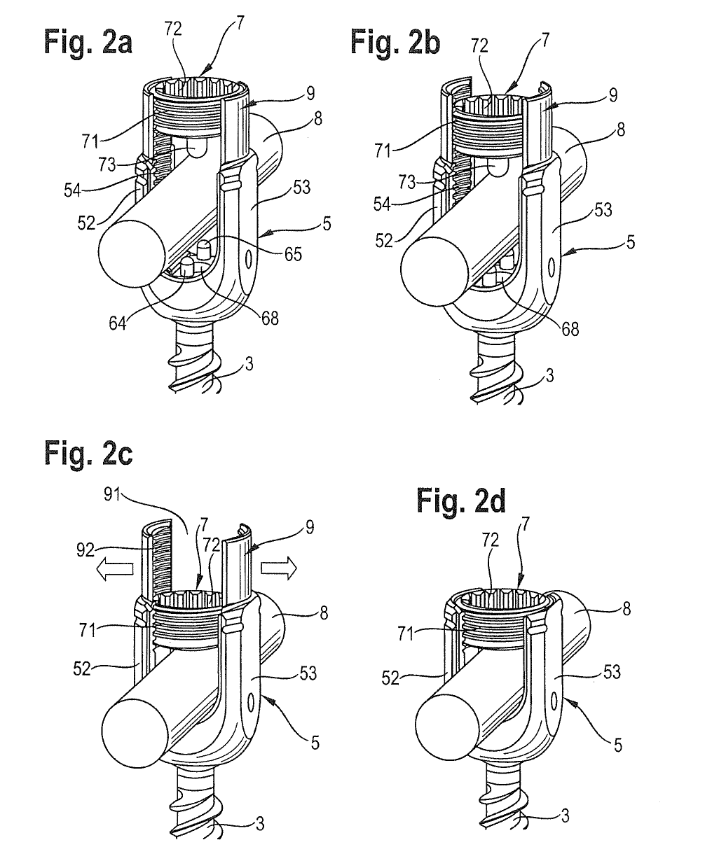

In use, as can be seen from FIGS. 2a to 4d, firstly at least two bone anchoring devices 1 are screwed into adjacent vertebrae, for example into the pedicles. At least one of the bone anchoring devices 1 is formed according to the invention. Thereafter, the rod 8 is inserted into the receiving part 5 and is fixed. The procedure of fixation is now explained with reference to FIGS. 2a to 4d. As shown in FIGS. 2a, 3a, 4a, in a first configuration the locking element 7 is screwed into the tube-shaped extension 9. The rod 8 is inserted into the channel 68 until it rests onto the pins 64, 65 of the pressure element 6 or on the pin 73 of the locking element 7. As can be seen from FIGS. 2b, 2c, 3b, 3c, 4b, 4c, when the locking element 7 is screwed in completely between the legs of the tube-shaped extension 9 and then between the legs 52, 53 of the receiving part 5, the pins 64, 65 are pressing into the surface of the rod 8 until they are fully immersed in the surface of the rod 8 due to the local flow of the material of the rod 8, which leads to a local elastic and/or plastic deformation of the rod 8. The engagement of the pins 64, 65 with the rod 8 is such that the integral structure of the rod 8 is not violated.

The pin 73 of the locking element 7 is also fully immersed in the surface of the rod 8 due to the local flow of the material of the rod 8 which leads to a local elastic and/or plastic deformation of the rod 8.

Due to the mechanical stop by the upper edge of the pressure element 6, the movement of the locking element 7 is restricted.

After screwing in the full immersion of the pins 64, 65, 73, the tube-shaped extension 9 is broken away via the predetermined breaking point after tightening the locking element 7 as indicated in FIGS. 2c, 3c by the arrows.

The dimension of the pins 64, 65, 73, in particular their height, the diameter and the radius of the free end portion is designed such that under a given pressure force which is limited by the stop described above, the pins 64, 65, 73 do not violate the integral structure of the rod 8.

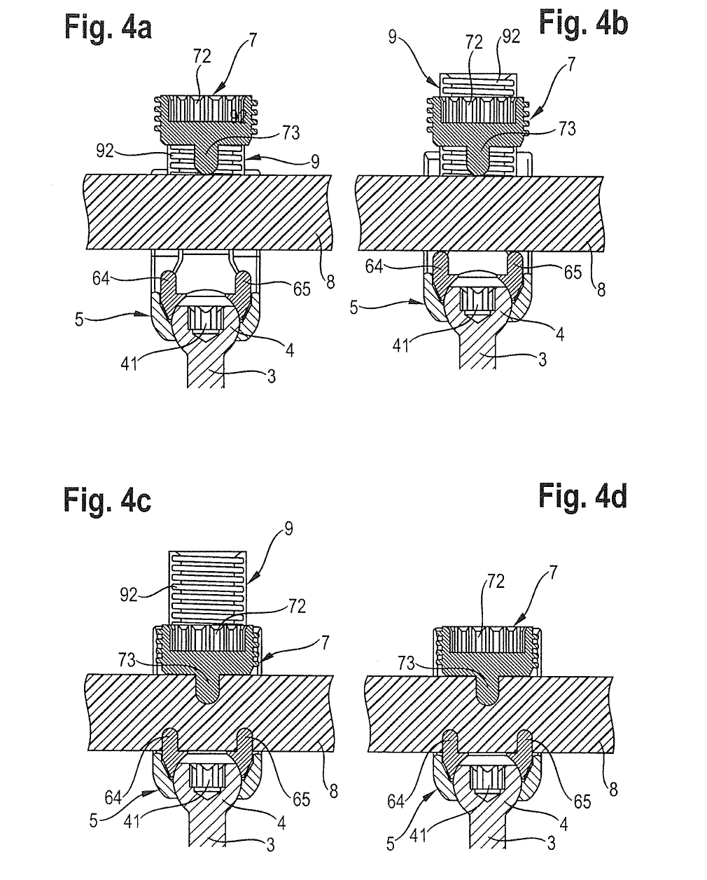

As can be seen in particular in FIGS. 4c, 4d, the arrangement of the pin fixation seen in the direction perpendicular to the longitudinal axis of the rod 8 is a three-point fixation, which is particularly safe. That means, there is no clamping on locations which are exactly on opposite sides of the rod 8 which may cause the danger of violating the integral structure of the rod 8 at the clamping site.

Several modifications are conceivable. For example, the number of pins in the bottom of the channel of the pressure element or also the number of pins of the locking element may vary. In some cases more than two pins might be of advantage referring to the pressure element. The shape of the pins can also vary. However, the height of the pins and the radius of the uppermost rounded portion must be designed such that there is no violation of the integral structure of the rod, while simultaneously providing safe fixation.

All other kinds of polyaxial bone anchoring assemblies known may be conceivable which can be modified so as to have the pins described above. For example, a polyaxial screw, where the bone anchor is inserted from below, a so-called bottom loader, may be also used.

1. A bone anchoring assembly for dynamic stabilization, comprising:
a bone anchoring element having a head and a shank to be anchored in a bone or a vertebra;
a rod having a flexible section which is at least partly made of a polymer material,
a receiving part for receiving the rod, the receiving part comprising a first channel with an approximately U-shaped cross-section with two free legs;
a pressure element; and
a monolithic locking element configured to directly cooperate with the legs to simultaneously secure the rod in the first channel and exert a pressure on the pressure element that exerts pressure onto the head of the bone anchoring element to lock the angular position of the bone anchoring element relative to the receiving part, wherein
a first pin-shaped projection is provided at the locking element which comes into contact with the flexible section of the rod when tightening the locking element.

2. The bone anchoring assembly according to claim 1, wherein the bone anchoring element and the receiving part are pivotably connected.

3. The bone anchoring assembly according to claim 1, wherein the pressure element comprises a second channel with an approximately U-shaped cross-section.

4. The bone anchoring assembly according to claim 4, wherein at least one second pin-shaped projection is provided in the second channel which comes into contact with the rod (8) when tightening the locking element.

5. The bone anchoring assembly according to claim 5, wherein the first and second pin-shaped projection is a longitudinal rod-shaped pin and the surface of the first and second pin-shaped projection which contacts the rod, is rounded.

6. The bone anchoring assembly according to claim 5, wherein a plurality of second pin-shaped projections is arranged on the bottom of the second channel.

7. The bone anchoring assembly according to claim 7, wherein the second pin-shaped projections are arranged along a line, which is parallel to the longitudinal axis of the rod.

8. The bone anchoring assembly according to claim 1, wherein the locking element is a screw with an external thread engaging the legs of the receiving part.

9. The bone anchoring assembly according to claim 7, wherein the second pin-shaped projections are provided at both outer ends of the second channel.

10. The bone anchoring assembly according to claim 1, wherein a stop is provided which limits the insertion of the locking element into the receiving part.

11. The bone anchoring assembly according to claim 7, wherein two second pin-shaped projection are provided in an axial offset to the first pin-shaped projection in such a manner that a three-point fixation is achieved.

12. The bone anchoring assembly according to claim 1, wherein a tube-shaped extension is provided which is integrally formed with the receiving part.

13. The bone anchoring assembly according to claim 13, wherein the tube-shaped extension can be broken away from the receiving part, preferably via a predetermined breaking point.

14. The bone anchoring assembly according to claim 1, wherein the rod is made at least partly of an elastomer material.

15. The bone anchoring assembly according to claim 1, wherein in the tightened state the projection is immersed in the flexible section of the rod without violating the integrity of a surface of the flexible section of the rod where the projection is immersed.

16. A method of attaching a bone anchoring assembly to a bone or vertebra, the bone anchoring assembly comprising a bone anchoring element having a head and a shank to be anchored in a bone or a vertebra, a rod having a flexible section which is at least partly made of a polymer material, a receiving part for receiving the rod, the receiving part comprising a first channel with an approximately U-shaped cross-section with two free legs; a pressure element; and a monolithic locking element configured to directly cooperate with the legs to simultaneously secure the rod in the first channel and exert a pressure on the pressure element that exerts pressure onto the head of the bone anchoring element to lock the angular position of the bone anchoring element relative to the receiving part, wherein a first pin-shaped projection is provided at the locking element which comes into contact with the flexible section of the rod when tightening the locking element, the method comprising:
attaching the bone anchoring element to a bone or vertebra;
inserting the rod in the receiving part;
and tightening the locking element to cause the pin-shaped projection of the locking element to immerse in the flexible section of the rod without violating the integrity of the surface of the flexible section of the rod to further secure the rod in the channel.

17. A bone anchoring assembly for dynamic stabilization, comprising:
a bone anchoring element having a head and a shank to be anchored in a bone or a vertebra;
a rod having a flexible section which is at least partly made of a polymer material,
a receiving part for receiving the rod, the receiving part comprising a first channel with an approximately U-shaped cross-section with two free legs; and
a monolithic locking element configured to directly cooperate with the legs to secure the rod in the first channel, wherein
a first pin-shaped projection is provided at the locking element which comes into contact with the flexible section of the rod when tightening the locking element,
wherein in a tightened state the projection is immersed in the flexible section of the rod without violating the integrity of a surface of the flexible section of the rod where the projection is immersed.
